(19) United States
(12) Patent Application Publication
(10) Pub. No.: US 2008/0009007 A1
(43) Pub. Date: Jan. 10, 2008

(54) CONTROLLED INITIATION OF PRIMER EXTENSION

(75) Inventors: John Lyle, Redwood Shores, CA (US); Paul Peluso, Hayward, CA (US); Gene Shen, San Jose, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: Pacific Biosciences of California, Inc., Menlo Park, CA (US)

(21) Appl. No.: 11/763,746

(22) Filed: Jun. 15, 2007

Related U.S. Application Data
(60) Provisional application No. 60/814,433, filed on Jun. 16, 2006.

(51) Int. Cl. C12Q 1/68

(52) U.S. Cl. 435/6

(57) ABSTRACT

Controlled initiation of primer extension in determination of nucleic acid sequence information by incorporation of nucleotides or nucleotide analogs. Preferred aspects include photo-initiated extension through the use of photo-cleavable blocking, groups on termini of primer sequences followed by non-terminating primer extension using nucleotides or nucleotide analogs that are not extension terminators.

### CROSS-REFERENCE TO RELATED APPLICATIONS

This application claims priority to Provisional U.S. Patent Application No. 60/814,433, filed on Jun. 16, 2006, the full disclosure of which is incorporated herein in its entirety for all purposes.

### STATEMENT REGARDING FEDERALLY SPONSORED RESEARCH

Not Applicable.

### BACKGROUND OF THE INVENTION

In a large number of analytical reactions, the ability to precisely control reaction parameters is critical. This includes not only controlling basic parameters like pH, temperature, and the chemical composition of the reaction, but also control over the initiation, termination and even location of the reaction.

In nucleic acid analyses that are based upon detection of polymerase mediated incorporation of nucleotides, control of the initiation of primer extension and the location of the reaction can be very useful. The present invention provides these and other benefits.

### BRIEF SUMMARY OF THE INVENTION

In particular, the present invention provides methods and compositions that are useful in controlling initiation of polymerase mediated primer extension reactions that may be broadly useful, but which are particularly useful in identifying sequence elements of the template nucleic acid. The control of initiation not only provides temporal control over initiation, but, when used in conjunction with optically confined reaction regions, also spatially controls such initiation.

In a first aspect, the invention provides a method of identifying a base in a nucleic acid template. The method comprises providing a polymerase/template/primer complex, wherein the primer comprises a removable blocking group at its 3′ terminus. The removable blocking group is removed to permit template dependent extension of the primer. One or more unprotected nucleotides or nucleotide analogs is then added to the primer to extend the primer in a template dependent manner, and the one or more added nucleotides or nucleotide analogs added to the primer are identified, thereby identifying a base in the nucleic acid template.

The invention also provides compositions that comprise a polymerase/template/primer complex, wherein the primer comprises a 3′ terminus protected with a photoremovable blocking group, and at least a first unprotected nucleotide or nucleotide analog.

### BRIEF DESCRIPTION OF THE DRAWINGS

FIG. 1 is a schematic illustration of the activatable primer extension initiation processes of the present invention.

FIG. 2 provides a schematic illustration of optically confined regions.

FIG. 3 schematically illustrates initiation of primer extension within an optical confinement using photo-deprotection of the primer sequence.

FIG. 4 illustrates a synthesis scheme for providing reversibly blocked nucleic acids for use in the invention.

### DETAILED DESCRIPTION OF THE INVENTION

The present invention is generally directed to activatable systems, methods and compositions for performing polymerase mediated, template dependent, primer extension reactions, and particularly performing such reactions in methods for determining sequence information for the template sequence using detection of nucleotides or nucleotide analogs incorporated onto the primer (or into the nascent strand).

The present invention provides a system for polymerase mediated, template dependent nucleic acid synthesis with controlled initiation, and particularly controlled initiation substantially only within a desired analytical zone. By controlling the initiation of the overall synthesis reaction, one can prevent adverse effects of random initiation or initiation throughout a given reaction mixture, including portions of the mixture that are not being analyzed. Such uncontrolled reaction can yield a variety of adverse effects upon the analyzed reaction region, such as generation of reaction by-products that may interfere with the reaction or the monitoring of that reaction, generation of partially visible reaction components, consumption of reagents, and the like.

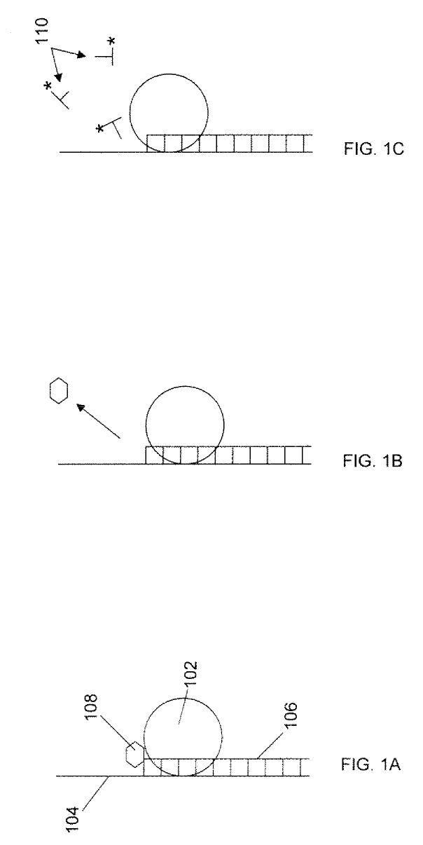

A general schematic illustration of the overall system of the present invention is illustrated in FIG. 1. As shown in panel A, a nucleic acid polymerase 102 is provided complexed with a template nucleic acid 104 and a complementary primer sequence 106. The primer sequence is provided blocked or capped at the 3′ terminus so as to prevent initiation of template dependent primer extension by blocking group 108. As shown in panel B, blocking group 108 is removed from the primer sequence. Presentation of the complex with an appropriate nucleotide or nucleotide analog 110, e.g., complementary to the adjacent base in template sequence 104, as shown in Panel C, then results in template dependent, polymerase mediated extension of the primer sequence.

A variety of removable blocking groups are known in the art for capping the 3′ hydroxyl group of a terminal base in a primer sequence, and include chemically removable groups, such as those used in solid or liquid phase nucleic acid synthesis methods (e.g., as described in U.S. Pat. Nos. 4,415,732; 4,458,066; 4,500,707; 4,668,777; 4,973,679; and 5,132,418; 4,725,677 and Re. 34,069).

As noted herein however, in the context of the present invention, photoremovable blocking groups are preferred. In particular, use of photoremovable groups allows for removal of the blocking groups without introducing new chemicals to the reaction system, and also allows for the focused activation of the system, as discussed in greater detail below. A number of different types of photoremovable chemical blocking groups have been described in the art. In general, such groups include, e.g., nitroveratryl, 1-pyrenylmethyl, 6-nitroveratryloxycarbonyl, dimethyldimethoxybenzyloxycarbonyl, 2-nitrobenzyloxycarbonyl, methyl-6-nitropiperonyloxycarbonyl, 2-oxymethylene anthraquinone, dimethoxybenzyloxy carbonyl, 5-bromo-7-nitroindolinyl, o-hydroxy-alpha-methyl cinnamoyl, and mixtures thereof, the compositions and applications of which are described in, e.g., U.S. Pat. Nos. 5,412,087, 5,143,854, 6,881,836, Albert et al., Nucl. Acids Res. (2003) 31(7):e35, Beier et al., Nucleic Acids Res. (2000) 28(4):e11, Pon et al, Nucleic Acids Res. (2004) 32(2):623-631, Olejnik et al., Nucleic Acids Res. (1998) 26(15):3572-3576, and Blanc et al. J. Org. Chem. (2002) 67:5567-5577, each of which is incorporated herein by reference in its entirety for all purposes.

In some cases, it will be desirable to employ photolabile blocking groups that are labile at the same wavelength of light used for analysis, e.g., excitation wavelengths, so that a single illumination system may be employed both for initiation of extension and for analysis during extension. However, in many cases, it may be desirable to separate the activation illumination from the analysis illumination, e.g., to avoid continued activation over time during analysis, that might lead to interference with the analysis. Depending upon the analysis wavelength(s), one may readily select from the variety of available protecting groups based upon their labile wavelengths.

For example, for those aspects of the invention that would benefit from the use of longer wavelengths for deprotection/extension initiation, appropriate longer wavelength labile groups would be used, such as brominated 7-hydroxycoumarin-4yl-methyls, which are photolabile at around 740 nm. Other such groups are known to those of skill in the art.

Also useful are such photolabile groups for coupling to alcohols, including, e.g., some of the groups described above, as well as p-nitrobenzyloxymethyl ether, p-methoxybenzylether, p-nitrobenzylether, mono, di or trimethoxytrityls, diphenylmethylsilyl ether, sisyl ether, 3′,5′-dimethoxybenzoincarbonate, methanesulfate, tosylate, and the like. These and a variety of other photocleavable groups may be employed in conjunction with this aspect of the invention, and are described in, e.g., the CRC Handbook of Organic Photochemistry and Photobiology, Second Edition, and Protective Groups in Organic Synthesis (T. W. Greene and P. G. Wuts, 3^{rd} Ed. John Wiley & Sons, 1999), each of which is incorporated herein by reference in its entirety for all purposes.

As noted previously, in addition to advantages of controlling the reaction, the present invention provides additional advantages of selecting for initiation of synthesis only in those portions of a reaction mixture where one is observing the reaction, and not elsewhere. In particular, the present invention provides for removal of the blocking group on the primer sequence within the analysis region of the reaction mixture. In one particularly preferred aspect, this is accomplished by using a photoremovable blocking group in an analysis that utilizes excitation radiation that performs the dual functions of removing the photoremovable protecting group and exciting fluorescent labeling groups on incorporated nucleotides or nucleotide analogs. Further, because one can relatively precisely direct that electromagnetic radiation, one can effectively initiate synthesis is a very small portion of the overall reaction mixture.

While direction of the excitation radiation may be accomplished through a variety of conventional focusing optics, that may provide illumination spots that are less than 10 μm in diameter, it will be appreciated that for a number of applications, the portion of a reaction mixture that is desired to be illuminated (also referred to as the illumination volume) and analyzed will be substantially smaller than such illumination spots may afford. Accordingly, in preferred aspects, the invention employs optically confined reaction regions, where an illumination volume can be further restricted.

Optically confined analysis regions may be achieved in a variety of different ways. For example, by using total internal reflectance microscopy, one can provide a very thin layer of illumination on an opposing side of a transparent substrate. Stated briefly, directing light at a transparent substrate at an angle that results in total internal reflection of the light beam will still yield some propagation of light beyond the substrate that decays exponentially over a very short distance, e.g., on the order of nanometers. By illuminating a reaction mixture on a substrate using total internal reflection through the substrate, one can effectively confine illumination to a very thin layer of the reaction mixture adjacent to the substrate, thereby providing an optically confined reaction region or volume.

Alternatively, one may use other optical confinement techniques, such as zero mode waveguides to provide optically confined regions of a reaction mixture. Briefly described, a zero mode waveguide typically includes a transparent substrate that has an opaque cladding layer deposited upon its surface. The cladding layer may be a variety of different types of opaque materials, including semiconductors, opaque polymers, metal films or the like. In particularly preferred aspects, metal films and more preferably, aluminum of chrome films are used as the cladding layer.

A small aperture or core is disposed through the cladding layer to the underlying transparent substrate. The core has a cross sectional dimension, e.g., diameter if circular, or width, if elongated, that prevents light that has a frequency below a cut-off frequency from propagating through the core. Instead, the light penetrates only a very short distance into the waveguide core when illuminated from one end, e.g., from below the transparent substrate, and that light decays exponentially as a function of distance from the entrance to the core. Typically, such waveguide cores have a cross sectional dimension of between about 10 and 200 nm, with preferred sizes being from about 20 to about 100 nm in cross sectional dimension, e.g., diameter of circular waveguides or width of linear or elongate waveguides. The result of illumination of such structures is a very small well in which a very small region proximal to the illuminated end of the core, is sufficiently illuminated (for activation and/or excitation), while the remainder of the core and any material therein, is not sufficiently illuminated. Zero mode waveguides, zero mode waveguide arrays, and their use in analytical applications are described in, e.g., U.S. Pat. Nos. 6,917,726, 7,013,054, and published U.S. Patent Application No. 2006-0061754, the full disclosures of which are hereby incorporated by reference for all purposes.

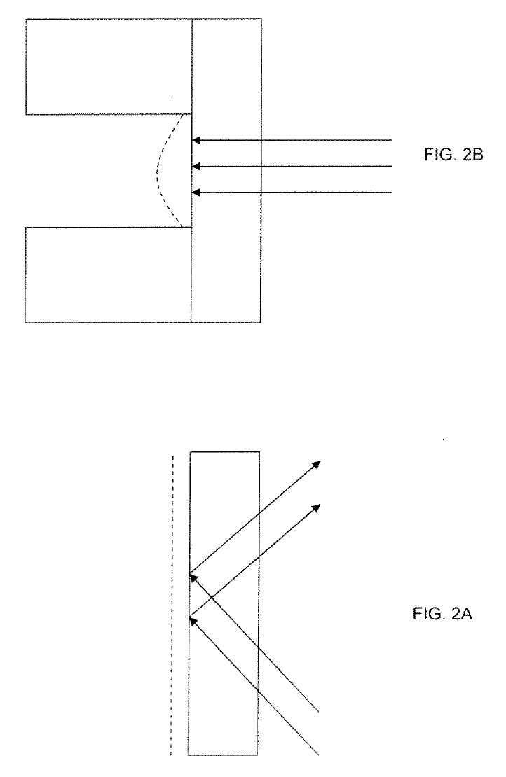

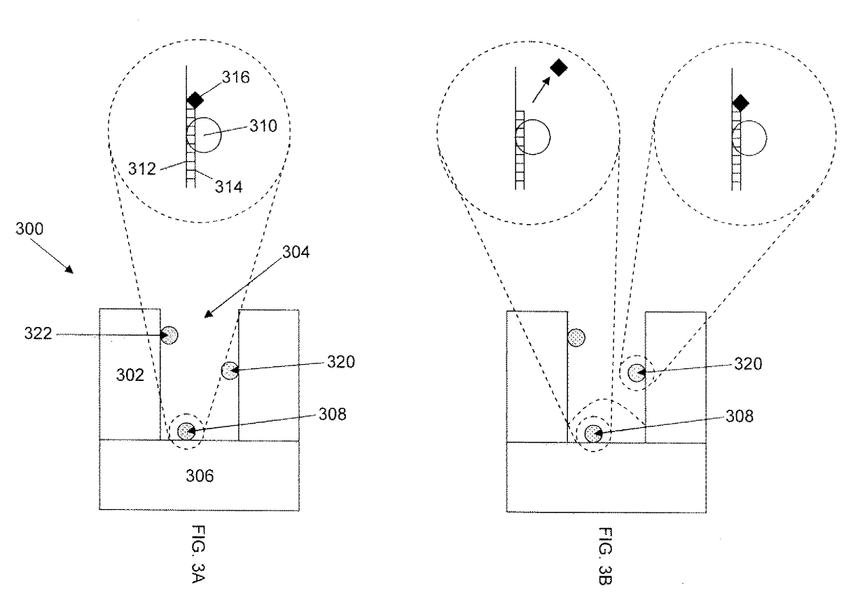

illustrations of optically confined regions are provided in FIG. 2. As shown in panel A, a substrate is illuminated using total internal reflection, resulting in a thin illumination region at the substrate's surface, as indicated by the dashed line over the substrate surface. In contrast, a zero mode waveguide, shown in Panel B, provides a small reaction region or volume proximal to the underlying substrate surface, and is further confined by the cladding layer, again as illustrated by the dashed line within the core of the zero mode waveguide structure.

By providing for an optically activatable system, one can further enhance the application of the system by selecting for active complexes that fall within the optically accessible portion of the analytical system. Rephrased, by only activating complexes that fall within an illumination region of a substrate, one ensures that only those complexes within the illuminated region are active, and thus reduce any interference from active complexes that are outside the illuminated region. Similar concepts have been described for immobilization within optically confined regions by optically activating coupling groups only within the optically confined region, e.g., within an illumination volume of a zero mode waveguide (See, e.g., commonly assigned U.S. patent application Ser. No. 11/394,352, filed Mar. 30, 2006, which is incorporated herein by reference in its entirety for all purposes).

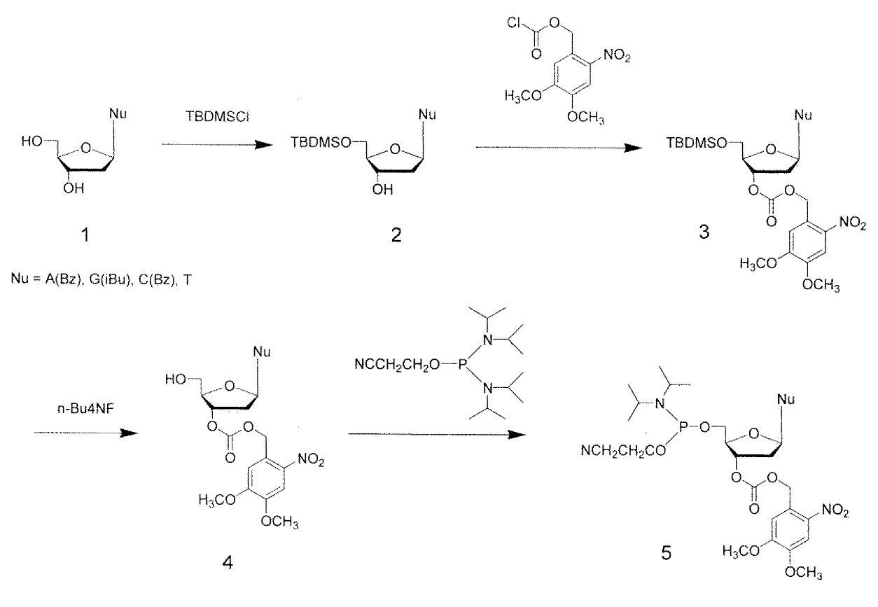

This advantage is schematically illustrated in FIG. 3, with respect to a zero mode waveguide. As shown in panel A, a zero mode waveguide 300 including a cladding layer 302 and a core 304 disposed through the cladding layer to the underlying substrate 306 is provided. A nucleic acid synthesis complex 308, is provided immobilized within the core (a number of different complexes 320 and 322 are also shown). The complex 308, shown in expanded view, includes a polymerase enzyme 310, a template sequence 312 and a primer sequence 314 bearing a 3′ terminal photoremovable blocking group 316. As shown in Panel B, illumination of the waveguide results in creation of a small illumination region or volume at the bottom of the core, as indicated by dashed line 318. The selective illumination then deprotects only the complexes within the illumination region, e.g., complex 308, and not complexes that are outside of the illumination region, e.g., complexes 320 (as shown in expanded view) and 322. The deprotection of the primer sequence in complex 308 then allows for primer extension, and ultimately as set forth below, detection of incorporated nucleotides.

A general synthetic approach for the preparation of the primer 314 bearing a 3′ terminal photoremovable blocking group 316 can be achieved by the use of the reverse (5′→3′) phosphoramidites in the oligonucleotide synthesis. The reverse phosphoramidite oligonucleotide synthesis has been widely used in the preparation of antisense oligos and other area (chemistries and syntheses generally available from, e.g., Link Technologies).

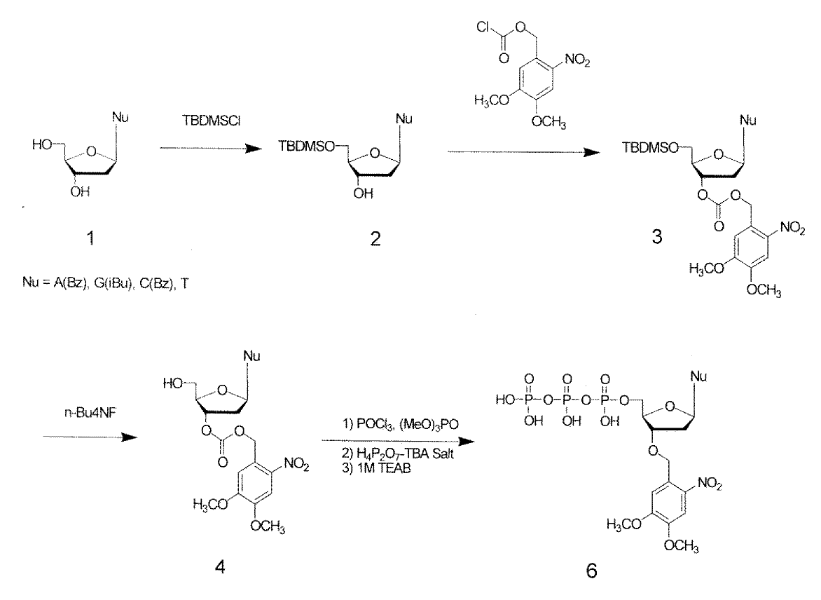

The synthetic scheme for the preparation of the phosphoramidite base unit with a photoremovable blocking group is outlined in the following synthetic scheme that is also illustrated in FIG. 4. The properly protected nucleoside 1 (Nu=A(Bz), G(iBu), C(Bz), T) is treated with tert-butyldimethylsilyl chloride (TBDMSCI) to give the selectively 5′-OH protected silyl ether 2. Reaction of the silyl ether 2 with 4,5-dimethyl-2-nitrobenzyl chlormate gives the carbonate 3. Deprotection of the silyl protection group on 3 with tetra-n-butylammonium floride gives the alcohol 4, which is then reacted with cyanoethyl tetrapropylphosphordiamitite to give the phosphitylated nucleotide 5.

Incorporation of the phosphitylated nucleotide 5 as the last base unit with the standard solid phase automated reverse phosphoramidite oligonucleotide synthesis chemistry can then provide the targeted primer with a photoremovable blocking group. These and related syntheses are discussed in, e.g., Albert et al., Nucl. Acids Res. (2003) 31(7):e35, and Claeboe et al., Nucleic Acids Res. (2003) 31(19):5685-5691, the full disclosures of which are incorporated herein by reference in their entirety for all purposes.

Alternatively, the corresponding nucleotide triphosphate with a photoremovable blocking group at the 3′-OH position can be synthesized as outlined in FIG. 5. Following the similar synthetic scheme as shown in FIG. 4 for the preparation of the 3′-protected alcohol 4, the alcohol 4 is then reacted with phosphorus oxychloride (POCl₃) and pyrophosphate to give the triphosphate nucleotide 6.

Incorporation of the triphosphate nucleotide 6 as the last base unit call be achieved enzymatically using a DNA polymerase to give the targeted primer with a photoremovable blocking group.

As noted above, while the systems of the invention will have a variety of applications where controlled initiation of primer extension is desired, it is particularly useful in controlled initiation of primer extension when used in conjunction with the identification of one or more bases in the template sequence based upon incorporation of nucleotides or nucleotide analogs. In particularly preferred aspects, ‘real time sequencing by incorporation’ is the desired application, where one detects each incorporated nucleotide as it is being incorporated into the nascent strand of primer extension. Examples of such sequencing by incorporation are described in, e.g., U.S. Pat. Nos. 7,033,764 and 7,052,847, the full disclosures of which are incorporated herein by reference for all purposes. For example, in some eases, nucleotide analogs bearing a fluorescent labeling group on a terminal phosphate group are incorporated into a growing nascent strand in a polymerase mediated, template dependent fashion at the complex. Upon incorporation, enhanced retention of the analog within the illumination region allows for identification of the incorporated base. Upon incorporation, the phosphate group attached to the nucleotide, and as a result, the labeled terminal phosphate group, are cleaved from the nucleotide and permitted to diffuse out of the illumination region. Because of the enhanced retention of the incorporated analog as compared to randomly diffusion analogs within the illuminated region, one can identify that incorporation. Terminal phosphate labeled nucleotide analogs and related compounds are described, for example in: U.S. Pat. Nos. 6,399,335 and 7,041,812; Published U.S. Patent Application Nos. 2003/0162213, 2004/0241716, 2003/0077610, 2003/0044781; and U.S. patent application Ser. No. 11/241,809 filed Sep. 29, 2005. In the context of the invention, only complexes that were initially deprotected will be able to perform primer extension reactions. Likewise, such extending complexes should primarily fall only within the illumination region that gave rise to their initial activation to begin with. The result is a double selection for the desired and analyzed activity, namely primer extension: (1) extension is only initiated within the illumination region; and (2) incorporation is only viewed within the illumination region.

In the context of sequence identification, the labeled nucleotides or nucleotide analogs will typically include fluorescent labeling groups that have distinguishable emission spectra, e.g., where each different type of base bears a detectable different fluorescent label. A variety of different fluorescent labeling groups are available from, e.g., Molecular Probes/Invitrogen (Eugene, Oreg.) or GE Healthcare, and include, e.g., the Alexa family of dyes and Cy family of dyes, respectively. In general such dyes, and their spectral characteristics are described in U.S. Pat. No. 7,041,812; Published U.S. Patent Application Nos. 2003/0162213, 2004/0241716, 2003/0077610, 2003/0044781; and U.S. patent application Ser. No. 11/241,809 filed Sep. 29, 2005, previously incorporated herein.

Although described in some detail for purposes of illustration, it will be readily appreciated that a number of variations known or appreciated by those of skill in the art may be practiced within the scope of present invention. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes.

What is claimed:

1. A method of identifying a base in a nucleic acid template, comprising:
providing a polymerase/template/primer complex, wherein the primer comprises a removable blocking group at its 3′ terminus;
removing the removable blocking group to permit template dependent extension of the primer; and
adding one or more unprotected nucleotides or nucleotide analogs to the primer to extend the primer in a template dependent manner;
identifying the one or more added nucleotides or nucleotide analogs added to the primer, and thereby identifying a base in the nucleic acid template.

2. The method of claim 1, wherein the removable blocking group comprises a photoremovable blocking group.

3. The method of claim 2, wherein the photoremovable blocking group is selected from the group of nitroveratryl, 1-pyrenylmethyl, 6-nitroveratryloxycarbonyl, dimethyldimethoxybenzyloxycarbonyl, 2-nitrobenzyloxycarbonyl, methyl-6-nitropiperonyloxycarbonyl, 2-oxymethylene anthraquinone, dimethoxybenzyloxy carbonyl, 5-bromo-7-nitroindolinyl, o-hydroxy-alpha-methyl cinnamoyl, and mixtures thereof.

4. The method of claim 1, wherein the polymerase/template/primer complex is immobilized upon a solid support.

5. The method of claim 1, wherein the identifying step comprises identifying individual unprotected nucleotides or nucleotide analogs as they are added to the primer.

6. The method of claim 5, wherein the individual nucleotide or nucleotide analogs are identified by optical characteristics.

7. The method of claim 6, wherein the optical characteristics comprise fluorescent molecules, each type of nucleotide or nucleotide analog bearing a detectably different fluorescent molecule.

8. The method of claim 7, wherein the fluorescent molecules are attached to the nucleotides or nucleotide analogs at a gamma phosphate or more distal phosphate from a nucleoside portion of the nucleotide or nucleotide analog.

9. The method of claim 1, wherein the polymerase/template/primer complex is immobilized in an optically confined region.

10. The method of claim 9, wherein the polymerase/template/primer complex is immobilized upon a surface of a transparent substrate and the optically confined region encompasses the surface using total internal reflection microscopy.

11. The method of claim 9, wherein the polymerase/template/primer complex is immobilized within an illumination volume of a zero mode waveguide.

12. A composition, comprising:
a polymerase/template/primer complex, wherein the primer comprises a 3′ terminus protected with a photoremovable blocking group; and
at least a first unprotected nucleotide or nucleotide analog.

13. The composition of claim 12, wherein the at least first unprotected nucleotide or nucleotide analog comprises a fluorescently labeled nucleotide or nucleotide analog.

14. The composition of claim 13, wherein the fluorescently labeled nucleotide or nucleotide analog comprises a phosphate labeled nucleotide or nucleotide analog.

15. The composition of claim 14, wherein the phosphate labeled nucleotide or nucleotide analog comprises a fluorescent label on a gamma phosphate or more distal phosphate from a nucleoside portion of the nucleotide or nucleotide analog.
